(19) United States
(12) Patent Application Publication
(10) Pub. No.: US 2004/0219194 A1
(43) Pub. Date: Nov. 4, 2004

(54) Transdermal drug delivery system for oxybutynin

(76) Inventors: Matthias Finckh, Munich (DE); Katalin Tisa-Bostedt, Pullach (DE); Eva Fischbacher, Bad Feilnbach (DE)

Correspondence Address: FROMMER LAWRENCE & HAUG, 745 FIFTH AVENUE- 10TH FL., NEW YORK, NY, 10151, US

(21) Appl. No.: 10/680,956

(22) Filed: Oct. 8, 2003

(30) Foreign Application Priority Data
Nov. 4, 2002 (DE) DE 102 51 256.6

(51) Int. Cl. A61K 9/70; A61F 13/00; A61K 35/78

(52) U.S. Cl. 424/449; 424/744

(57) ABSTRACT

The invention concerns a transdermal drug delivery system (TDS) comprising

a cover which is impermeable for the active ingredient,

a matrix containing oxybutynin as active ingredient and

a facultative release liner, wherein the matrix further comprises

an Aloe Vera extract,

a facultative skin care agent,

a pressure sensitive adhesive and

a cross linking agent for the adhesive.

WO 99/48 493 describes an oxybutynin patch obtained according to the socalled hot melt process. It is stated that the patch does not contain any enhancer. Nevertheless substances which are usually used as enhancers, are mentioned, especially citric acid triester.

U.S. Pat. No. 5,601,839 describes triacetin as an agent improving permeability.

As regards oxybutynin patches, U.S. Pat. No. 5,411,740 and WO 93/23 025 should also be mentioned.

The problem underlaying the invention is solved by a transdermal drug delivery system (TDS) comprising

a cover which is impermeable for the active ingredient,

a matrix containing oxybutynin as active ingredient and

a facultative release liner, wherein the matrix further comprises

an Aloe Vera extract,

a pressure sensitive adhesive and

a cross linking agent for the adhesive.

The transdermal drug delivery system according to the invention may comprise racemic oxybutynin, R-oxybutynin, S-oxybutynin or desethyl-oxybutynin.

Further, the pressure sensitive adhesive of the transdermal drug delivery system according the invention may comprise or consist of an acrylate based polymer, preferably a polymer based on an acrylate-vinyl acetate copolymer.

Further, the pressure sensitive adhesive of the transdermal drug delivery system according to the invention may comprise or consist of Durotak 2287 or Durotak 2516.

Further, the matrix of the transdermal drug delivery system according to the invention may comprise Ti-acetylacetonate, Al-acetylacetonate or polybutyl-titanate as crosslinking agent.

Further the extracting agent of the Aloe Vera-extract of the transdermal drug delivery system according to the invention may be a vegetable oil, preferably soybean oil.

An Aloe Vera-extract is available from, for example, Caesar & Loretz (Hilden/Germany).

Further, the Aloe Vera-extract of the transdermal drug delivery system according to the invention may comprise 5 to 15% by weight of Aloe Vera oil and 95 to 85% by weight of the vegetable oil.

Further, the matrix of the transdermal drug delivery system according to the invention may comprise the Aloe Vera-extract as the only enhancer.

Further, the matrix of the transdermal drug delivery system according to the invention may comprise 5 to 40, preferably 10 to 35 and especially 15 to 30% by weight of oxybutynin (based on the matrix).

Further, the matrix of the transdermal drug delivery system according to the invention may comprise 10 to 25, preferably 12 to 20 and especially 14 to 18% by weight of Aloe Vera-extract (based on the matrix).

Further, the matrix of the transdermal drug delivery system according to the invention may comprise 0.1 to 5.0, preferably 0.3 to 3 and especially 0.5 to 2.0% by weight of the crosslinking agent (based on the matrix).

The transdermal drug delivery system according to the invention may have a surface of 5 to 80, preferably 10 to 60 and especially 20 to 50 cm².

### EXAMPLE AND COMPARATIVE EXAMPLE

A composition of a matrix according to the invention was provided as follows:

| | |
|---|---|
| Oxybutynin | 20.0% |
| Aloe Vera-extract (soy bean oil) | 15.0% |
| Ti-acetylacetonate (Tyzor AA 75) | 1.3% |
| Durotak 2287 | remainder |

This composition was subjected to a permeation test (mouse skin). The maximum flux was 9.2 μg/cm²/h. The permeation was 190 μg/cm²/24 h.

According to U.S. Pat. No. 5,601,839 a matrix was provided with the following composition

| | |
|---|---|
| Oxybutynin | 20.0% |
| Triacetin | 15.0% |
| Al-Acetylacetonate | 0.5% |

Durotak 2051 (Acrylate/Vinylacetate adhesive) remainder This composition was also subjected to a permeation test (mouse skin). The maximum flux was 5.3 μg/cm³/h. The permeation was 80 μg/cm²/24 h.

1. Transdermal drug delivery system (TDS) comprising
a cover which is impermeable for the active ingredient,
a matrix containing oxybutynin as active ingredient and
a facultative release liner, wherein the matrix further comprises
an Aloe Vera extract,
a pressure sensitive adhesive and
a cross linking agent for the adhesive.

2. Transdermal drug delivery system according to claim 1, comprising racemic oxybutynin, R-oxybutynin, S-oxybutynin or desethyl-oxybutynin.

3. Transdermal drug delivery according to claim 1, wherein the pressure sensitive adhesive of the matrix is comprised of an actrylate based polymer, preferably a polymer based on an acrylate-vinyl acetate copolymer.

4. Transdermal drug delivery system according to claim 1, wherein the matrix is comprised of Durotak 2287 or Durotak 2516.

5. Transdermal drug delivery system according to claim 1, wherein the matrix comprises Ti-acetylacetonate, Al-acetylacetonate or polybutyl-titanate as crosslinking agent.

6. Transdermal drug delivery system according to claim 1, wherein the extracting agent of the Aloe Vera-extract is a vegetable oil, preferably soybean oil.

7. Transdermal drug delivery system according to claim 6, wherein the Aloe Vera-extract comprises 5 to 15% by weight of Aloe Vera oil and 95 to 85% by weight of vegetable oil.

8. Transdermal drug delivery system according to claim 1, wherein the matrix comprises the Aloe Vera-extract as the only enhancer.

9. Transdermal drug delivery system according to claim 1, wherein the matrix comprises 5 to 40, preferably 10 to 35 and especially 15 to 30% by weight of oxybutynin (based on the matrix).

10. Transdermal drug delivery system according to claim 1, wherein the matrix comprises 10 to 25, preferably 12 to 20 and especially 14 to 18% by weight of Aloe Vera-extract (based on the matrix).

11. Transdermal drug delivery system according to claim 1, wherein the matrix comprises 0.1 to 5.0, preferably 0.3 to 3 and especially 0.5 to 2.0% by weight of the crosslinking agent (based on the matrix).

12. Transdermal drug delivery system according to claim 1, wherein the system has a surface of 5 to 80, preferably 10 to 60 and especially 20 to 50 cm².
